(19) United States
(12) Patent Application Publication
(10) Pub. No.: US 2022/0378998 A1
(43) Pub. Date: Dec. 1, 2022

(54) PROTECTION COVER FOR ANASTOMOTIC PART

(71) Applicants: Akeo HAGIWARA, Otsu-shi (JP); KANEKA CORPORATION, Osaka (JP)

(72) Inventor: Akeo HAGIWARA, Otsu-shi (JP)

(73) Assignees: Akeo HAGIWARA, Otsu-shi, Shiga (JP); KANEKA CORPORATION, Osaka-shi, Osaka (JP)

(21) Appl. No.: 17/884,112

(22) Filed: Aug. 9, 2022

Related U.S. Application Data
(63) Continuation of application No. PCT/JP2021/004062, filed on Feb. 4, 2021.

(30) Foreign Application Priority Data
Feb. 14, 2020 (JP) 2020-023831

(51) Int. Cl. A61M 1/36; A61B 17/11

(52) U.S. Cl. CPC A61M 1/3655; A61B 17/11; A61B 2017/1107

(57) ABSTRACT

A protection cover (1) for an anastomotic part in which a first blood vessel is joined to a second blood vessel, having a tubular portion (2) that is configured to cover an outside of the first blood vessel, and a planar portion (4) that is configured to cover an outside of the second blood vessel, the protection cover (1) comprising a knitted fabric which is formed from continuous yarn extending from the tubular portion (2) to the planar portion (4).

### TECHNICAL FIELD

The present invention relates to a protection cover for a vascular anastomotic part, and for example, relates to a protection cover that can be installed in an arteriovenous shunt.

### BACKGROUND ART

For dialysis patients, dialysis treatment is regularly carried out, where blood is taken from a patient's body to remove waste products, excess water, minerals and others by a dialysis apparatus, and then returned to the patient's body. In hemodialysis, it is necessary to form a vascular access that serves as a gateway for blood to be taken out of a blood vessel and returned to the blood vessel, and for that, an arteriovenous shunt that connects an artery and a vein to allow a large amount of blood to pass through may be prepared on the patients forearm or the like. In such an arteriovenous shunt, for example, an end-to-side anastomotic part is formed in which an end of a vein is joined to a side of an artery.

In an arteriovenous shunt, when excessive force is applied to the anastomotic part, or the flow of blood passing through the anastomotic part is disturbed, or a sudden change in blood pressure on the venous side or in blood circulation status is occurred, an excessive pressure change to the blood vessel wall occurs in the anastomotic part or vein on the downstream side thereof; and as a result, excessive stress is generated in the blood vessel wall, leading to pathological intima thickening, that may cause obstruction or stenosis. As a countermeasure for that, it has been proposed to install a protection cover at an anastomotic part of a blood vessel. For example, Patent Literature 1 discloses an apparatus for configuring first and second blood vessels connected by an anastomosis, comprising a coupler and a sleeve, wherein: the coupler comprises a mount having a saddle-like shape that seats on and couples to the first blood vessel, an adapter having a substantially cylindrical shape that extends from the mount, and a brace extending from a side of the mount that can be closed to prevent the coupler from lifting off from the first blood vessel; and a sleeve is connected to the adapter that ensheathes and holds a portion of the second blood vessel so that the first and second blood vessels are joined at an acute angle. Patent Literature 2 discloses an external vascular support for forming a junction between an artery and a vein anastomosed to the artery, comprising an arterial portion connected to an artery and a venous portion connected to a vein, wherein the arterial portion and the venous portion are connected at an acute angle being formed rounded to form a filleted junction between the vein and the artery and maintain the vein at an acute angle to the artery.

### CITATION LIST

### Patent Literature

### Patent Literature 1

U.S. Pat. No. 10,299,794

### Patent Literature 2

Japanese Unexamined Patent Publication No. 2018-126556

### SUMMARY OF INVENTION

### Technical Problem

As described above, various protection covers for a vascular anastomotic part have been conventionally proposed, and on the anastomotic part, it is desired that the anastomotic part is stably held while the protective cover does not excessively restrain movement of the blood vessel. The present invention has been made in view of the above circumstances, and an object of the present invention is to provide a protection cover that is capable of stably holding an anastomotic part while preventing excessive restraint of movement of a blood vessel at the anastomotic part.

### Solution to Problem

The protection cover of the present invention which solves the above problems is a protection cover for an anastomotic part in which a first blood vessel is joined to a second blood vessel, comprising: a tubular portion that is configured to cover an outside of the first blood vessel; and a planar portion connected to the tubular portion, the planar portion configured to cover an outside of the second blood vessel, wherein the protection cover comprises a knitted fabric which is formed from continuous yarn extending from the tubular portion to the planar portion.

Since a knitted fabric is excellent in elasticity and flexibility, the protection cover formed from a knitted fabric can stably hold an anastomotic part without excessively restraining the first blood vessel and the second blood vessel. Therefore, the protection cover appropriately holds the first blood vessel so as to extend in a desired direction starting from the second blood vessel, and can control pressure and pulsation of blood at the anastomotic part as desired. In addition, the protection cover prevents damage to blood vessels and surrounding tissues. Further, since the protection cover comprises a knitted fabric continuous from the tubular portion to the planar portion, the anastomotic part is less likely to be subjected to localized undue force when the protective cover is attached to the anastomotic part. In the case where the anastomotic part is an arteriovenous shunt, the tubular part composed of the knitted fabric appropriately presses a vein, that is the first blood vessel, whereby pressure and pulsation of arterial blood can be gradually suppressed from the anastomotic part to the vein, and an excessive change in blood flow of the vein in an early stage of formation of the anastomotic part can be alleviated.

It is preferable that the tubular portion has a larger elongation stress in an axial direction than in a circumferential direction. When the tubular portion is configured in this manner, flexibility of the first blood vessel is less likely to be inhibited.

It is preferable that the tubular portion and each of the planar portion does not have a sewn part. By configuring the protection cover in this manner, elasticity and flexibility of the entire protection cover can be appropriately maintained.

It is preferable that the planar portion is formed in a substantially rectangular shape or an oval shape. In addition, it is preferable that the planar portion is formed in a substantially rectangular shape including shorter sides and longer sides, and the planar portion is configured to cover the outside of the second blood vessel so that the shorter sides of the planar portion extend substantially parallel to an extending direction of the second blood vessel.

It is preferable that the tubular portion has a portion including an end opening portion located opposite to a connection portion at which the tubular portion is connected to the planar portion, and the tubular portion is configured so that an inner diameter of the portion including the end opening increases as a distance from the planar portion increases. The tubular portion may be configured that the tubular portion has a portion including an end opening portion located opposite to a connection portion at which the tubular portion is connected to the planar portion, and the tubular portion is configured so that an inner diameter of the portion including the end opening increases such that an angle of the tubular portion with respect to an axial direction of the tubular portion increases as a distance from the planar portion increases. The tubular portion may be configured so as to comprise: a first expanded portion which is formed widened as a distance from the planar portion increases, at an angle A with respect to an axial direction of the tubular portion; and a second expanded portion which is located farther from the planar portion than the first expanded part, and is formed widened as the distance from the planar portion increases, at an angle B with respect to the axial direction of the tubular portion larger than the angle A, and the tubular portion has a largest diameter at an end opening portion opposite to a connection portion at which the tubular portion is connected to the planar portion.

A non-tubular extension portion may be provided at an end portion of the tubular portion located opposite to a connection portion at which the tubular portion is connected to the planar portion. In this case, it is preferable that the non-tubular extension portion comprises a knitted fabric which is formed from continuous yarn extending from the tubular portion to the extension portion.

The planar portion may have a first edge and a second edge, wherein the first edge may be separated into two or more, and the second edge may be separated into two or more.

The planar portion may have a cut edge formed by cutting the knitted fabric, and the planar portion may be provided with a reinforce part along the cut edge. The planar portion may have a first edge and a second edge, and the planar portion may be provided with reinforce parts along the first edge and the second edge, respectively, and another reinforce part positioned between the reinforce parts along the first edge and the reinforce part along the second edge.

The planar portion may have four or more cut edges formed by cutting the knitted fabric, extending in a radial direction of the tubular portion from a connection with the tubular portion.

### Advantageous Effects of Invention

According to the protection cover of the present invention, an anastomotic part can be stably held without excessively restraining the first blood vessel and the second blood vessel. Therefore, the protection cover appropriately holds the first blood vessel so as to extend in a desired direction starting from the second blood vessel, and can control pressure and pulsation of blood at the anastomotic part as desired. In addition, the protection cover prevents damage to blood vessels and surrounding tissues. Further, since the protection cover comprises a knitted fabric continuous from the tubular portion to the planar portion, the anastomotic part is less likely to be subjected to localized undue force when the protective cover is attached to the anastomotic part. In the case where the anastomotic part is an arteriovenous shunt, the tubular part composed of the knitted fabric appropriately presses a vein, that is the first blood vessel, whereby pressure and pulsation of arterial blood can be gradually suppressed from the anastomotic part to the vein, and an excessive change in blood flow of the vein in an early stage of formation of the anastomotic part can be alleviated.

### BRIEF DESCRIPTION OF DRAWINGS

FIG. 1 represents an embodiment of a protection cover of the present invention, and shows a perspective view of the protection cover.

FIG. 2 shows a perspective view of the protection cover shown in FIG. 1, that is attached to a vascular anastomotic part.

FIG. 3 represents another embodiment of a protection cover of the present invention, and shows a perspective view of the protection cover.

FIG. 4 represents another embodiment of a protection cover of the present invention, and shows a perspective view of the protection cover.

FIG. 5 represents another embodiment of a protection cover of the present invention, and shows a perspective view of the protection cover.

FIG. 6 represents another embodiment of a protection cover of the present invention, and shows a perspective view of the protection cover.

FIG. 7 represents another embodiment of a protection cover of the present invention, and shows a perspective view of the protection cover.

FIG. 8 represents another embodiment of a protection cover of the present invention, and shows a perspective view of the protection cover.

FIG. 9 represents another embodiment of a protection cover of the present invention, and shows a perspective view of the protection cover.

FIG. 10 represents another embodiment of a protection cover of the present invention, and shows a perspective view of the protection cover.

FIG. 11 represents another embodiment of a protection cover of the present invention, and shows a perspective view of the protection cover.

### DESCRIPTION OF EMBODIMENTS

Hereinafter, the present invention is specifically explained below based on the following embodiments; however, the present invention is not restricted by the embodiments described below of course, and can be certainly put into practice after appropriate modifications within in a range meeting the gist of the above and the below, all of which are included in the technical scope of the present invention. In the drawings, hatching or a reference sign for a member may be omitted for convenience, and in such a case, the description and other drawings should be referred to. In addition, sizes of various members in the drawings may differ from the actual sizes thereof, since priority is given to understanding the features of the present invention.

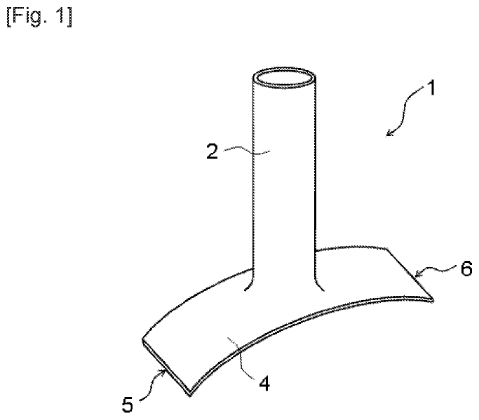

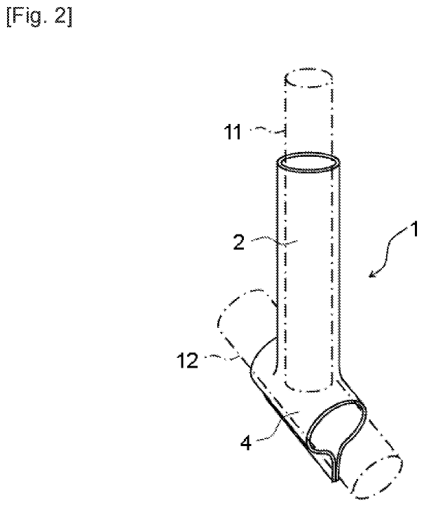

FIGS. 1 and 2 show an embodiment of a protection cover of the present invention. FIG. 1 shows a perspective view of the protection cover before the protection cover is attached to a vascular anastomotic part, and FIG. 2 shows a perspective view of the protection cover in the state where the protection cover is attached to a vascular anastomotic part. In FIG. 2, a first blood vessel and a second blood vessel are shown by a one dot chain line.

A protection cover 1 is an object to be attached to an anastomotic part where a first blood vessel 11 is joined to a second blood vessel 12, and comprises a tubular portion 2 that is configured to cover an outside of the first blood vessel 11 and a planar portion 4 that is configured to cover an outside of the second blood vessel 12. The first blood vessel 11 and the second blood vessel 12 may be an artery, a vein or an artificial blood vessel. Examples of the anastomotic part include an end-to-side anastomotic part where an end of the first blood vessel is joined to a side of the second blood vessel, and a side-to-side anastomotic part where a side of the first blood vessel is joined to a side of the second blood vessel; and the drawing shows an example of application to an end-to-side anastomotic part where an end of the first blood vessel 11 is joined to a side of the second blood vessel 12. For example, in dialysis patients, an end-to-side anastomotic part in which an end of a vein is joined to a side of an artery is formed, where an arteriovenous shunt is provided as a vascular access that serves as a gateway for blood to be taken out of a blood vessel, dialyzed and returned to the blood vessel. The protection cover 1 can be attached to the anastomotic part by covering an outside of the first blood vessel 11 by the tubular portion 2, and winding the planar portion 4 around the second blood vessel 12 so as to cover an outside of the second blood vessel 12.

The tubular portion 2 is a portion that covers the first blood vessel 11, and has an axial direction and a circumferential direction. The axial direction of the tubular portion 2 corresponds to an extending direction of the first blood vessel 11 when the protection cover 1 is attached to the anastomotic part. The tubular portion 2 has an end opening located opposite to a portion at which the tubular portion 2 is connected to the planar portion 4. The lengths in the axial direction and the circumferential direction of the tubular portion 2 are appropriately set according to the size of the first blood vessel 11, and for example, the length in the axial direction can be set in the range of 3 mm to 150 mm and the length in the circumference direction can be set in the range of 4 mm to 60 mm. When forming the anastomotic part, the first blood vessel 11 is inserted into the tubular portion 2 before joining the first blood vessel 11 to the second blood vessel 12.

The planar portion 4 is a portion that covers the second blood vessel 12, and is formed in a planar shape. The planar portion 4 is connected to the tubular portion 2 and is configured to cover an outside of the second blood vessel 12. When forming the anastomotic part, the first blood vessel 11 is joined to the second blood vessel 12 by suturing or the like, and then the planar portion 4 is wound around the second blood vessel 12, so that the second blood vessel 12 is covered with the planar portion 4. One side part and the other side part of the planar portion 4 wound around the second blood vessel 12 are joined to each other by sewing or the like, whereby the position of the planar portion 4 is fixed around the second blood vessel 12. The size of the planar portion 4 is appropriately set according to the size of the second blood vessel 12, and for example, the length in the extending direction of the second blood vessel 12 can be set in the range of 3 mm to 50 mm, and the length in the circumferential direction of the blood vessel 12 can be set in the range of 5 mm to 70 mm.

The protection cover 1 is configured to comprise a knitted fabric which is formed from continuous yarn extending from the tubular portion 2 to the planar portion 4. That is, the protection cover 1 comprises a knitted fabric continuous from the tubular portion 2 to the planar portion 4. Since a knitted fabric is excellent in elasticity and flexibility, the protection cover 1 formed from a knitted fabric can stably hold the anastomotic part without excessively restraining the first blood vessel 11 and the second blood vessel 12. Therefore, the protection cover 1 appropriately holds the first blood vessel 11 so as to extend in a desired direction starting from the second blood vessel 12, and can control pressure and pulsation of blood at the anastomotic part as desired. In addition, the protection cover 1 prevents damage to blood vessels and surrounding tissues. Further, since the protection cover 1 comprises a knitted fabric continuous from the tubular portion 2 to the planar portion 4, the anastomotic part is less likely to be subjected to localized undue force when the protective cover 1 is attached to the anastomotic part. In the case where the anastomotic part is an arteriovenous shunt, the tubular part 2 composed of the knitted fabric appropriately presses a vein, that is the first blood vessel 11, whereby the pressure and pulsation of arterial blood can be gradually suppressed from the anastomotic part to the vein, and an excessive change in blood flow of the vein in an early stage of formation of the anastomotic part can be alleviated.

The protection cover 1 does not have a sewn part between the tubular portion 2 and the planar portion 4, and preferably, each of the tubular portion 2 and the planar portion 4 also does not have a sewn part. For example, it is preferable that the tubular portion 2 does not have a sewn part extending in the axial direction, and the yarn constituting the knitted fabric extends continuously in the circumferential direction of the tubular portion 2, that is, the yarn constituting the knitted fabric extends spirally while forming knitted stitches. In particular, it is preferable that the entire protection cover 1 is composed of single knitted fabric, that is, the entire protection cover 1 is preferably composed of a knitted fabric formed from continuous yarn. By configuring the protection cover 1 in this manner, elasticity and flexibility of the entire protection cover 1 can be appropriately maintained.

The type of the knitted fabric is not particularly limited, and may be warp knitting or weft knitting. Examples of knitting texture of the warp knitting include half knitting, back half knitting, quinz coat knitting, and satin knitting. The weft knitting includes circular knitting and flat knitting, and examples of knitting texture of the weft knitting include plain knitting, rib knitting, double side knitting, miilan-rib knitting, and jacquard knitting. In view of excellent in elasticity, the knitted fabric is preferably composed of a weft knitted fabric.

In the case where the knitted fabric is composed of a weft knitted fabric, the knitted fabric can be manufactured using a circular knitting machine or a flat knitting machine. As the flat knitting machine, it is preferable to use a wholegarment (registered trademark) knitting machine. By using such a knitting machine, it becomes easy to form the tubular portion 2 and the planar portion 4 without a sewn part and continuously form the tubular portion 2 to the planar portion 4 without a sewn part.

The yarn constituting the knitted fabric is preferably composed of a resin having biocompatibility, and examples the resin include synthetic resins such as, for example, a polyolefin resin (e.g., polyethylene and polypropylene), a polyamide resin (e.g., nylon), a polyester resin (e.g., polyethylene terephthalate), an aromatic polyether ketone resin (e.g., PEEK), a polyether polyamide resin, a polyurethane resin, a polyimide resin, a fluororesin (e.g., PTFE, PFA and ETFE), a polyvinyl chloride resin and a silicone resin. The yarn constituting the knitted fabric can also be composed of a resin used for artificial blood vessels (e.g., polyester, PTFE, polyurethane). The yarn constituting the knitted fabric may be biodegradable.

The shape of the planar portion 4 is not particularly limited, and it is preferable that the planar portion 4 has a first edge 5 and a second edge 6 provided so as to face each other. When the planar portion 4 is configured in this manner, by winding the planar portion 4 around the blood vessel 12 so that the first edge 5 and the second edge 6 extend substantially parallel to the extending direction of the second blood vessel 12, a part of the planar portion 4 on the first edge 5 side and a part of that on the second edge 6 side can be easily joined by sewing or the like. In this case, it is preferable that the first edge 5 and the second edge 6 have straight portions extending substantially parallel to each other.

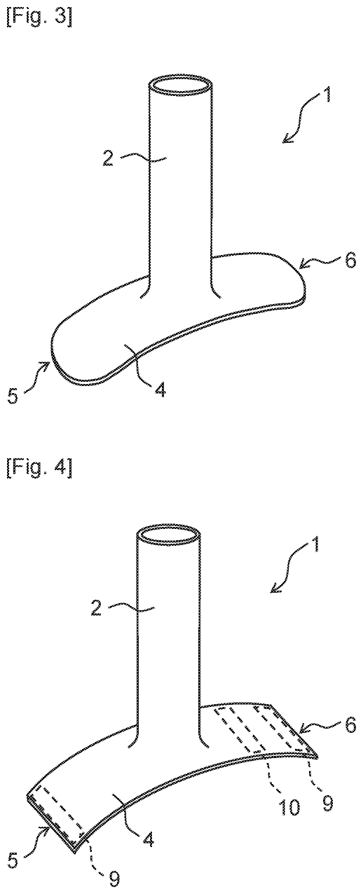

It is preferable that the planar portion 4 is formed in a substantially rectangular shape, such as shown in FIG. 1, or an oval shape, such as shown in FIG. 3. The substantially rectangular shape includes a rectangular shape with rounded corners. The oval shape includes an elliptic shape, a shape in which two semicircles are connected by straight lines, such as a track field shape, an egg-like shape, and the like. When the planar portion 4 is formed in such a shape, it becomes easy to perform an operation of winding the planar portion 4 around the second blood vessel 12, and it becomes easy to secure a long joining length in joining a part of the planar portion 4 on the first edge 5 side and a part of that on the second edge 6 side by sewing or the like.

It is preferable that the planar portion 4 has a short direction, and the short direction extends substantially parallel to the extending direction of the second blood vessel 12. The short direction of the planar portion 4 means a direction that gives the shortest length in the planar portion 4. Specifically, it is preferable that the planar portion 4 is formed in a substantially rectangular shape including shorter sides and longer sides, and the planar portion 4 is configured to cover the outside of the second blood vessel 12 so that the shorter sides of the planar portion 4 extend substantially parallel to an extending direction of the second blood vessel 12. When the planar portion 4 is configured in this manner, an area of the second blood vessel 12 covered with the planar portion 4 is reduced when winding the planar portion 4 around the second blood vessel 12, and movement of the second blood vessel 12 is less likely to be restrained by the protection cover 1. In addition, since a surgical field can be made smaller, it is less invasive. The short direction of the planar portion 4 is preferably perpendicular to the direction from the first edge 5 side to the second edge 6 side. Further, it is preferable that the planar portion 4 is formed in a substantially rectangular shape or an oval shape, and an extending direction of a short side of the substantially rectangular shape or a short axis direction of the oval shape corresponds to the short direction.

It is preferable that the planar portion 4 is composed of single knitted fabric. The planar portion 4 may be formed into a desired shape by adjusting the way of knitting, or the planar portion 4 may be formed into a desired shape by cutting a part of the knitted fabric after forming the knitted fabric. It is preferable that the planar portion 4 is formed by the former manner, that is, the planar portion 4 does not have a cut edge which is formed by cutting a part of the knitted fabric.

The planar portion 4 may be provided with a reinforce part in part or all along a peripheral edge thereof. As a result, the yarn of the knitted fabric constituting the planar portion 4 is less likely to fray, and handleability of the protection cover 1 is improved. The reinforce part can be formed by increasing knitting density of the knitted fabric of the reinforce part, applying a resin (for example, an adhesive) to the knitted fabric, attaching a resin film to the knitted fabric, or welding the knitted fabric by heat sealing, ultrasonic welding or the like. Among them, the reinforce part is preferably formed by a high-density part having a high knitting density of the knitted fabric, that makes it easy to secure flexibility of the reinforce part. In this case, the planar portion 4 has a high-density part formed with a higher knitting density and a low-density part formed with a lower knitting density.

In the case where the planar portion 4 has a cut edge formed by cutting the knitted fabric, it is also preferable that the reinforce part is provided along the cut edge. Thereby, the yarn of the knitted fabric constituting the planar portion 4 is less likely to fray starting from the cut edge.

FIG. 4 shows an example in which reinforce parts are provided on the planar portion 4 of the protection cover 1 shown in FIG. 1. As shown in FIG. 4, it is preferable that the reinforce parts 9 are provided at least along the first edge 5 and along the second edge 6. As a result, in sewing a part of the planar portion 4 on the first edge 5 side and a part of that on the second edge 6 side to each other, it becomes easy to firmly join them. The planar portion 4 may be provided with the reinforce parts 9 along the first edge 5 and the second edge 6, respectively, and another reinforce part 10 positioned between the reinforce parts 9 along the first edge 5 and the reinforce part along the second edge 6. As a result, even in the case where the second blood vessel 12 is thin, by sewing a part of the planar portion 4 on the first edge 5 side or the second edge 6 side and the another reinforce part 10 to each other, it becomes easy to firmly join them.

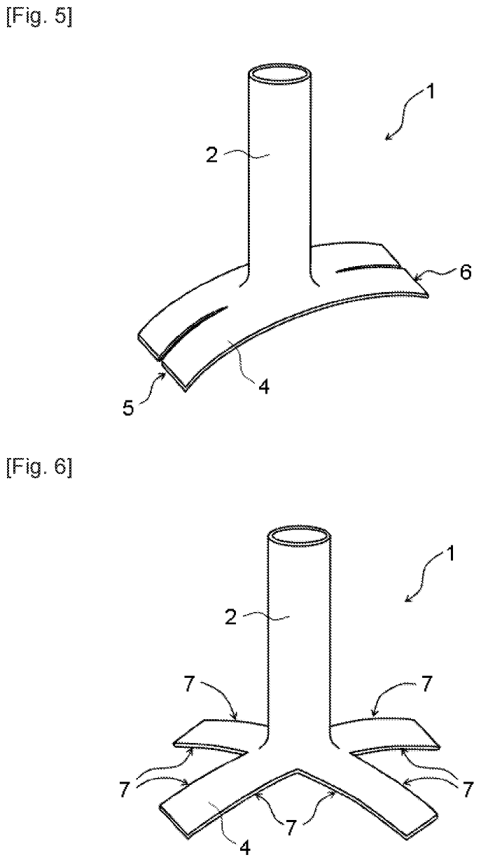

FIG. 5 shows another embodiment of the protection cover of the present invention. In the protection cover 1 shown in FIG. 5, the first edge 5 of the planar portion 4 is separated into two, and the second edge 6 is separated into two. That is, the planar portion 4 is separated into two starting from the first edge 5 and further separated into two stating from the second edge 6. In FIG. 5, the planar portion 4 has slits each extending from an edge of the planar portion 4 toward a joint portion at which the tubular portion 2 is connected to the planar portion 4, so that the planar portion 4 has strips. The first edge 5 may be separated into three or more, and the second edge 6 may also be separated into three or more. When the planar portion 4 is configured in this manner, the movement of the second blood vessel 12 is further less likely to be restrained by the planar portion 4. In addition, since the plurality of separated first edges 5 and the plurality of separated second edges 6 can be separately joined to each other by sewing or the like to form a plurality of joining parts, the planar portion 4 is less likely to shift with respect to the second blood vessel 12, and the planar portion 4 can be attached to the second blood vessel 12 more stably.

In the protection cover 1 shown in FIG. 5, the first edge 5 may be separated into two or more and the second edge 6 may be separated into two or more by making slits in the planar portion 4: or the planar portion 4 may be formed into a shape in which the first edge 5 is separated into two or more and the second edge 6 is separated into two or more by adjusting the way of knitting. In view of preventing the yarn of the knitted fabric constituting the planar portion 4 from fraying and ensuring flexibility of the planar portion 4, it is preferable that the planar portion 4 is formed into a desired shape by adjusting the way of knitting.

In view of easily manufacturing the protection cover 1, it is also possible to configure the tubular portion 2 and the planar portion 4 by forming a knitted fabric into a tubular shape and making two or more slits from one edge of the knitted fabric of the tubular shape. In this case, the planar portion 4 is formed separately into two or more, and the planar portion 4 has four or more cutting edges extending in a radial direction of the tubular portion 2 from the connection with the tubular portion 2. That is, for each slit, two cut edges that is formed by cutting the knitted fabric are formed. FIG. 6 shows an example of the protection cover thus formed. In the protection cover 1 shown in FIG. 6, the tubular portion 2 and the planar portion 4 are formed by making four slits from one edge of the knitted fabric formed in a tubular shape, and the planar portion 4 has eight cut edges 7 extending in a radial direction of the tubular portion 2 from the connection with the tubular portion 2. That is, the planar portion 4 has slits each extending from an edge of the planar portion 4 toward a joint portion at which the tubular portion 2 is connected to the planar portion 4, so that the planar portion 4 has strips each extending outwardly from the joint portion. The radial direction described here may be a direction extending away from the tubular portion 2 that starts from the connection between the planar portion 4 and the tubular portion 2. The number of cut edges 7 in this case is preferably 6 or more, more preferably 8 or more, and preferably 16 or less, more preferably 12 or less.

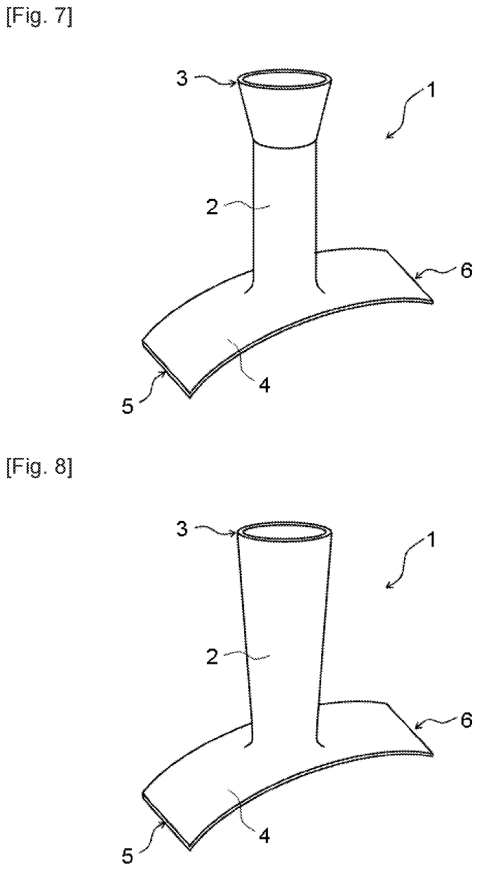

The tubular portion 2 may be configured so that the diameter is constant along the axial direction, or may be configured so that the diameter varies along the axial direction. In the protection covers 1 shown in FIGS. 1 to 6, the diameter of the tubular portion 2 is formed constant along the axial direction. On the other hand, as shown in FIGS. 7 to 10, the tubular portion 2 may be configured so that an end 3 located opposite to a side connected to the planar portion 4 has the largest diameter. That is, the tubular portion 2 may have a largest diameter at the end opening portion opposite to a connection portion at which the tubular portion 2 is connected to the planar portion 4. In this case, the tubular portion 2 has a diameter-expanded portion which is formed widened as a distance from the planar portion 4 increases. When the tubular portion 2 is configured in this manner, an operation of inserting the first blood vessel 11 into the tubular portion 2 can be easily conducted. In the case where the anastomotic part is an arteriovenous shunt, changes in a vein, that is the first blood vessel 11, can be smoothly supported.

As shown in FIGS. 7 to 10, the tubular portion 2 is preferably configured so that a portion including the end 3 located opposite to the side connected to the planar portion 4 is widened as a distance from the planar portion 4 increases. That is, it is preferable that the tubular portion 2 has a portion including the end opening portion located opposite to the connection portion at which the tubular portion 2 is connected to the planar portion 4, and the tubular portion 2 is configured so that an inner diameter of the portion including the end opening increases as a distance from the planar portion 4 increases. When the tubular portion 2 is configured in this manner, the first blood vessel 11 can be easily inserted into the tubular portion 2, and after attaching the protection cover 1 to the anastomotic part of a blood vessel, it becomes easy to conduct an operation of fixing the end of the tubular portion 2 whose diameter is widened to a tissue in the vicinity of the first blood vessel 11 by suturing or the like. As a result, after the protection cover 1 is attached to the anastomotic part of a blood vessel, the tubular portion 2 is easily fixed in position without being displaced from the first blood vessel 11. The tubular portion 2 may be configured to have an inner diameter so that the inner diameter increases as a distance increases away from the planar portion 4 toward the end opening.

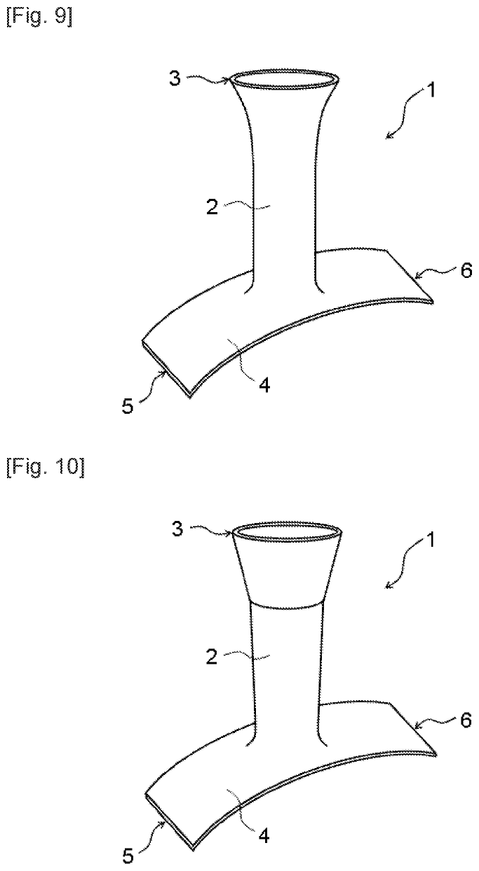

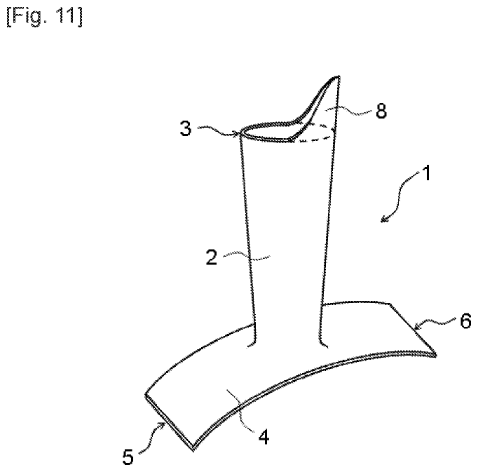

The portion where the diameter of the tubular portion 2 is widened may be formed in part or all of the tubular portion 2 with respect to the axial direction of the tubular portion 2. In FIGS. 7 and 9, only a part of the tubular portion 2, that is, the end 3 located opposite to the side connected to the planar portion 4, is configured so that the diameter thereof increases as a distance from the planar portion 4 increases. That is, only the end opening portion of the tubular portion 2 is formed so that an inner diameter of the tubular portion 2 increases as a distance from the planar portion 4 increases. Meanwhile, in FIGS. 8 and 10, the entire tubular portion 2 is configured so that the diameter thereof increases as a distance from the planar portion 4 increases. In either case, it is preferable that the end 3 located opposite to the side connected to the planar portion 4 has the largest diameter. At a part where the diameter of the tubular portion 2 is the largest, the length in the circumferential direction of the tubular portion 2 is preferably in the range of, for example, 15 mm to 60 mm. Meanwhile, at a part where the diameter of the tubular portion 2 is the smallest, the length in the circumferential direction of the tubular portion 2 is preferably in the range of, for example, 4 mm to 26 mm.

The tubular portion 2 may be configured so that a portion including the end 3 located opposite to the side connected to the planar portion 4 is widened such that an angle of the tubular portion 2 with respect to the axial direction increases as a distance from the planar portion 4 increases. That is, the tubular portion 2 may be configured so that an inner diameter of the portion including the end opening increases such that an angle of the tubular portion 2 with respect to an axial direction of the tubular portion 2 increases as a distance from the planar portion 4 increases. Also in the case where the tubular portion 2 is configured in this manner, the first blood vessel 11 can be easily inserted into the tubular portion 2, and after attaching the protection cover 1 to the anastomotic part of a blood vessel, it becomes easy to conduct an operation of fixing the end of the tubular portion 2 whose diameter is widened to a tissue in the vicinity of the first blood vessel 11 by suturing or the like.

FIGS. 9 and 10 show examples of the protection cover configured in this manner. In the protection cover 1 shown in FIG. 9, the tubular portion 2 is configured so that the portion including the end 3 located opposite to the side connected to the planar portion 4 is widened in a curved shape, viewed in a cross section along the axial direction of the tubular portion 2, as a distance from the planar portion 4 increases. In the protection cover 1 shown in FIG. 10, the tubular portion 2 is configured so that the portion including the end 3 located opposite to the side connected to the planar portion 4 is widened in linearly in multiple steps, viewed in a cross section along the axial direction of the tubular portion 2, as a distance from the planar portion 4 increases. Specifically, the protection cover 1 shown in FIG. 10 is configured that the tubular portion 2 comprises a first expanded portion which is formed widened as a distance from the planar portion 4 increases, at an angle A with respect to the axial direction of the tubular portion 2, and a second expanded portion which is located farther from the planar portion than the first expanded part, and is formed widened as a distance from the planar portion 4 increases, at an angle B with respect to the axial direction of the tubular portion 2 larger than the angle A.

The tubular portion 2 may be configured so as to extend substantially perpendicular to the planar portion 4, or may be configured so as to extend diagonally to the planar portion 4. The angle of the extending direction of the tubular portion 2 with respect to the planar portion 4 can be appropriately set according to a desired shape of the anastomotic part. For example, it is preferable that the tubular portion 2 extends from the planar portion 4 so as to form an angle of 30° or more and 90° or less with respect to the planar portion 4.

It is preferable that the tubular portion 2 has a larger elongation stress in the axial direction than in the circumferential direction. When the tubular portion 2 is configured in this manner, the tubular portion 2 easily extends along the circumferential direction, so that the pressure on the first blood vessel 11 by the protection cover 1 can be reduced, and flexibility of the first blood vessel 11 is less likely to be inhibited. For the tubular portion 2, for example, the elongation stress in the axial direction is preferably 1.05 times or more, more preferably 1.1 times or more, even more preferably 1.2 times or more, and preferably 10 times or less, more preferably 8 times or less, even more preferably 5 times or less of the elongation stress in the circumferential direction. The elongation stress of the tubular portion 2 is measured by extending the tubular portion 2 in the axial direction or the circumferential direction using a tensile tester. The elongation stress is determined by measuring the stress at 30% elongation. Specifically, the original fabric of the knitted fabric that constitutes the tubular portion 2 is cut out with a width of 1 inch to make a test piece, and both ends of the test piece are fixed to chucks of a tensile tester so that the gripping distance is 50 mm. The end of the test piece is pulled at a tensile speed of 200 mm/min, the stress when the distance between the chucks reaches 130 mm is measured, and the value at this time is taken as the elongation stress.

The tubular portion 2 may be provided with a reinforce part described above at the end 3 located opposite to the side connected to the planar portion 4. For example, the tubular portion 2 may be provided with a high-density part formed with a high knitting density of the knitted fabric as the reinforce part. By providing the reinforce part in the tubular portion 2, it becomes easy to conduct an operation of fixing the tubular portion 2 to a tissue in the vicinity of the first blood vessel 11 by suturing.

In the protection cover 1 shown in FIGS. 7 to 10, the shape of the planar portion 4 is not particularly limited. The protection cover 1 shown in FIGS. 7 to 10 may have, for example, the planar portion 4 as shown in FIG. 5 or FIG. 6.

As shown in FIG. 11, the protection cover 1 may be configured that a non-tubular extension portion 8 is provided at the end 3 of the tubular portion 2 on a side opposite to the side connected to the planar portion 4. The non-tubular extension portion 8 is provided at an end portion of the tubular portion 2 located opposite to the connection portion at which the tubular portion 2 is connected to the planar portion 4. By fixing the extension portion 8 to a tissue in the vicinity of the first blood vessel 11 by suturing or the like after attaching the protection cover 1 to the anastomotic part of a blood vessel, the tubular portion 2 can be prevented from being displaced with respect to the first blood vessel 11.

The extension portion 8 is preferably composed of a knitted fabric, and more preferably comprises a knitted fabric which is formed of continuous yarn extending from the tubular portion 2 to the extension portion 8. That is, it is preferable that the protection cover 1 comprises a knitted fabric continuous from the tubular portion 2 to the extension portion 8. By forming the extension portion 8 in this manner, flexibility of the protection cover 1 can be enhanced in the region from the tubular portion 2 to the extension portion 8.

The shape of the extension portion 8 is not particularly limited. The extension portion 8 may be formed into a desired shape by adjusting the way of knitting, or the extension portion 8 may be formed into a desired shape by cutting a part of the knitted fabric after forming the knitted fabric. It is preferable that the extension portion 8 is formed by the former manner, that is, the extension portion 8 does not have a cut edge which is formed by cutting a part of the knitted fabric. As a result, the yarn of the knitted fabric constituting the extension portion 8 is less likely to fray. In the case where the protection cover 1 has the extension portion 8, the shapes of the tubular portion 2 and the planar portion 4 are not particularly limited, and the tubular portion 2 and the planar portion 4 described above can be combined as desired.

The extension portion 8 may be provided with a reinforce part described above. For example, the extension portion 8 may be provided with a high-density part having a higher knitting density of the knitted fabric than the tubular portion 2, as the reinforce part. By providing the reinforce part in the extension portion 8, it becomes easy to conduct an operation of fixing the extension portion 8 to a tissue in the vicinity of the first blood vessel 11 by suturing.

This application claims priority to Japanese Patent Application No. 2020-023831, filed on Feb. 14, 2020. All of the contents of the Japanese Patent Application No. 2020-023831, filed on Feb. 14, 2020, are incorporated by reference herein.

### REFERENCE SIGNS LIST

1: protection cover
2: tubular portion
3: end opposite to a side connected to a planar portion
4: planar portion
5: first edge
6: second edge
7: cut edge
8: extension portion
9: reinforce part
10: another reinforce part
11: first blood vessel
12: second blood vessel

1. A protection cover for an anastomotic part in which a first blood vessel is joined to a second blood vessel, comprising:
a tubular portion that is configured to cover an outside of the first blood vessel; and
a planar portion connected to the tubular portion, the planar portion configured to cover an outside of the second blood vessel, wherein
the protection cover comprises a knitted fabric which is formed from continuous yarn extending from the tubular portion to the planar portion.

2. The protection cover according to claim 1, wherein
the tubular portion has a larger elongation stress in an axial direction than in a circumferential direction.

3. The protection cover according to claim 1, wherein
each of the tubular portion and the planar portion does not have a sewn part.

4. The protection cover according to claim 1, wherein
the planar portion is formed in a substantially rectangular shape or an oval shape.

5. The protection cover according to claim 1, wherein
the planar portion is formed in a substantially rectangular shape including shorter sides and longer sides, and
the planar portion is configured to cover the outside of the second blood vessel so that the shorter sides of the planar portion extend substantially parallel to an extending direction of the second blood vessel.

6. The protection cover according to claim 1, wherein
the tubular portion has a portion including an end opening portion located opposite to a connection portion at which the tubular portion is connected to the planar portion, and
the tubular portion is configured so that an inner diameter of the portion including the end opening increases as a distance from the planar portion increases.

7. The protection cover according to claim 1, wherein
the tubular portion has a portion including an end opening portion located opposite to a connection portion at which the tubular portion is connected to the planar portion, and
the tubular portion is configured so that an inner diameter of the portion including the end opening increases such that an angle of the tubular portion with respect to an axial direction of the tubular portion increases as a distance from the planar portion increases.

8. The protection cover according to claim 1, wherein
the tubular portion comprises:
a first expanded portion which is formed widened as a distance from the planar portion increases, at an angle A with respect to an axial direction of the tubular portion; and
a second expanded portion which is located farther from the planar portion than the first expanded part, and is formed widened as the distance from the planar portion increases, at an angle B with respect to the axial direction of the tubular portion larger than the angle A, and
the tubular portion has a largest diameter at an end opening portion opposite to a connection portion at which the tubular portion is connected to the planar portion.

9. The protection cover according to claim 1, wherein
a non-tubular extension portion is provided at an end portion of the tubular portion located opposite to a connection portion at which the tubular portion is connected to the planar portion.

10. The protection cover according to claim 9, wherein
the non-tubular extension portion comprises a knitted fabric which is formed from continuous yarn extending from the tubular portion to the extension portion.

11. The protection cover according to claim 1, wherein
the planar portion has a first edge and a second edge,
the first edge is separated into two or more, and
the second edge is separated into two or more.

12. The protection cover according to claim 1, wherein
the planar portion has a cut edge formed by cutting the knitted fabric, and
the planar portion is provided with a reinforce part along the cut edge.

13. The protection cover according to claim 1, wherein
the planar portion has a first edge and a second edge, and
the planar portion is provided with reinforce parts along the first edge and the second edge, respectively, and another reinforce part positioned between the reinforce parts along the first edge and the reinforce part along the second edge.

14. The protection cover according to claim 1, wherein
the planar portion has four or more cut edges formed by cutting the knitted fabric, extending in a radial direction of the tubular portion from a connection with the tubular portion.

15. The protection cover according to claim 1, wherein
the tubular portion has an end opening located opposite to a portion at which the tubular portion is connected to the planar portion, and
the tubular portion is configured to have an inner diameter so that the inner diameter increases as a distance increases away from the planar portion toward the end opening.

16. The protection cover according to claim 1, wherein
the planar portion has slits each extending from an edge of the planar portion toward a joint portion at which the tubular portion is connected to the planar portion, so that the planar portion has strips each extending outwardly from the joint portion.

17. A method for protecting an anastomotic part at which a first blood vessel is joined to a second blood vessel, comprising:
attaching the protection cover of claim 1 to the anastomotic part by covering an outside of the first blood vessel by the tubular portion, and winding the planar portion around the second blood vessel so as to cover an outside of the second blood vessel.
